(19) United States
(12) Patent Application Publication
(10) Pub. No.: US 2024/0366141 A1
(43) Pub. Date: Nov. 7, 2024

(54) DISSECTING BETA WAVEFORMS USING CONVOLUTIONAL DICTIONARY LEARNING ACROSS SENSORY PERCEPTION, AGING, AND DISEASE

(71) Applicant: Brown University, Providence, RI (US)

(72) Inventors: Stephanie Jones, Hingham, MA (US); David Zhou, Arlington, MA (US)

(21) Appl. No.: 18/656,157

(22) Filed: May 6, 2024

Related U.S. Application Data
(60) Provisional application No. 63/464,340, filed on May 5, 2023.

(51) Int. Cl. A61B 5/374; A61B 5/384

(52) U.S. Cl. CPC A61B 5/374; A61B 5/384; A61B 2503/40; A61B 2503/42

(57) ABSTRACT

Disclosed herein is a pattern recognition and feature extraction system and method configured to detect and analyze non-stationary, transient, or locally structured signals from longer time-series data of obtained beta waveforms of an animal to characterize a biomarker of a brain state or condition. An example system comprises a computing device configured to process obtained recordings of electrical activity arising from a brain of an animal for detecting beta wave events. A first representation of short-time segments may be generated to represent amplitude fluctuations indicating extrema and time-domain features in the beta wave events and a second representation for identifying temporal positions of the extrema in the first representation. The computing device compares statistical distributions of feature characteristics and generates mean waveforms of signals from at least one condition aligned by the temporal positions assigned to one of extracted feature types to indicate a brain state of the animal.

### CROSS REFERENCE TO RELATED APPLICATION

The application claims priority to U.S. Provisional Patent Application No. 63/464,340, filed May 5, 2023, entitled “DISSECTING BETA WAVEFORMS USING CONVOLUTIONAL DICTIONARY LEARNING ACROSS SENSORY PERCEPTION, AGING, AND DISEASE,” the contents of which are incorporated by reference herein in their entirety.

### GOVERNMENT LICENSE RIGHTS

This patent application was made with government support under grant numbers AG076227 and MH130415 awarded by the National Institutes of Health. The government has certain rights in this application.

### FIELD OF TECHNOLOGY

The present disclosure generally relates to beta waveforms characterization to reveal predictive biomarkers of sensory perception, aging, and disease, and more particularly relates to a system and method configured to analyze brain wave recordings (e.g., magneto- or electroencephalography (M/EEG) beta waveform features) using time-domain pattern recognition and feature extraction methods to characterize a biomarker of a brain state or condition.

### BACKGROUND

Neuronal activity in the brain of an animal gives rise to transmembrane currents that may be measured in the extracellular medium. Electric current contributions from all active cellular processes within a volume of brain tissue superimpose at a given location in the extracellular medium and generate a potential. V. (a scalar measured in Volts), with respect to a reference potential. The difference in Ve between two locations gives rise to an electric field (a vector whose amplitude is measured in Volts per distance) that is defined as the negative spatial gradient of Ve. Electric fields can be monitored by extracellularly placed electrodes with sub-millisecond time resolution and can be used to interpret many facets of neuronal communication and computation.

Electroencephalography (EEG) is one of the most widely used methods for the investigation of the electric activity of the brain. Magnetoencephalography (MEG) uses superconducting quantum interference devices (SQUIDs) to measure tiny magnetic fields outside the skull (typically in the 10-1,000 fT range) from currents generated by the neurons. Electrocorticography (ECoG) is another method for studying various cortical phenomena in clinical settings. It uses subdural platinum-iridium or stainless steel electrodes to record electric activity directly from the surface of the cerebral cortex, thereby bypassing the signal-distorting skull and intermediate tissue. EEG, MEG and ECOG mainly sample electrical activity that occurs in the superficial layers of the cortex. Electrical events at deeper locations may be explored by inserting metal or glass electrodes, or silicon probes into the brain to record the local field potential (LFP; also known as micro-EEG). Recording the wide-band signal (direct current to 40 kHz), which contains both action potentials and other membrane potential-derived fluctuations in a small neuronal volume, using a microelectrode yields the most informative signal for studying cortical electrogenesis. In addition, voltage changes may be detected by membrane-bound voltage-sensitive dyes or by genetically expressed voltage-sensitive proteins. Using the voltage-sensitive dye imaging (VSDI) method, the membrane voltage changes of neurons in a region of interest can be detected optically, using a high-resolution fast-speed digital camera, at the peak excitation wavelength of the dye.

In some examples, M/EEG non-invasively may be used as an indication of human brain activity with millisecond resolution providing reliable markers of healthy and disease states. Relating these macroscopic signals to underlying cellular and circuit-level generators is a limitation that constrains using M/EEG to reveal novel principles of brain function or to translate findings into new therapies for neuropathology. Beta rhythms or waves in human M/EEG measured brain activity have been implicated in a wide range of cognitive and disease processes. For example, M/EEG may be used to study the aging process in humans. Beta waves are prominent during states of concentration and problem solving. Beta waves are common in the M/EEG's of most waking adults but may also be present during drowsiness. Beta waves tend to be more visible in the M/EEG when a subject's eyes are open. Excessive beta activity can relate to symptoms of brain over-arousal such as anxiety, obsessiveness, sleep difficulties, hyperactivity, etc. On the other hand, deficient beta activity can relate to symptoms of brain under aroused such as difficulty in concentrating, problem solving, etc.

Further, neuro-degenerative diseases are characterized by the progressive dysfunction and loss of neurons in patients' brains. These diseases are often associated with the mis-folding and aggregation of aberrant proteins. Alzheimer's disease (AD), dementia, and Parkinson's disease (PD) are several examples of major neurodegenerative diseases. In one study, invasive single unit and local field potential recordings from the human basal ganglia (a group of subcortical nuclei found in the brains of vertebrates) showed that excessive and oscillatory synchronization occurs in the basal ganglia in PD patients in the beta frequency range.

Beta waves in human M/EEG measured brain activity manifested in distinct peaks on spectrograms may be found in various locations of the cortex in normal subjects. In the frequency between 13 and 30 Hz, beta waves are more often found in frontal or central areas when compared to posterior regions of the cortex. Certain features of the beta waveforms (e.g., time-domain features) may have predictive value that has not yet been extensively characterized.

Accordingly, there is a need for a data-driven system and method for detecting and analyzing M/EEG beta waveform features to reveal predictive biomarkers of neuronal mechanisms at a cellular and circuit level.

### SUMMARY

Among other features, the present disclosure provides a system deployed within a communication network for detecting and analyzing non-stationary, transient, or locally structured signals from longer time-series data of obtained beta waveforms to characterize a biomarker of a brain state or condition. In one embodiment, an example system comprising a first computing device, comprising: a non-transitory machine-readable storage medium storing instructions; and a processor coupled to the non-transitory computer-readable storage medium and configured to execute the instructions to: obtain recordings of electrical activity arising from a brain of at least one animal, detect beta wave events from the recordings by at least extracting extrema and time-domain features in unnormalized representations of the recordings, process the beta wave events to generate a first representation of short-time segments representing a plurality of amplitude fluctuations indicating the extrema and a second representation for identifying temporal positions of the extrema in the first representation, compare statistical distributions of a plurality of feature characteristics of the beta wave events based upon extracted extrema and time-domain features and the first and second representations, and generate mean waveforms of signals from at least one condition aligned by the temporal positions assigned to at least one of extracted feature types to indicate a brain state of the at least one animal.

In some embodiments, the system may further comprise a second computing device configured to collect the recordings of the at least one animal via a plurality of sensors, wherein the recordings comprise at least one of magneto- or electroencephalograph (M/EEG) recordings, local field potential (LFP) recordings, or electrocorticogram (ECoG) recordings.

In one aspect, the extrema and time-domain features may comprise characteristics selected from the group consisting of: a peak time, a trough time, a peak amplitude, a trough amplitude, an oscillation period, a peak width, a trough width, an inter-peak timing, and an inter-trough timing. The processor of the system may be further configured to execute the instructions to: determine empirical distributions of the characteristics; and simulate, via resampling, an effect of alterations of the empirical distributions of at least one of the characteristics on the mean waveforms.

In yet another embodiment, the processor of the first computing device may be configured to store normative data of waveform features of the beta wave events and empirical or probability distributions or likelihoods thereof, and employ one or more time-domain feature learning and extraction methods to perform prediction of clinical indication and functional brain states of the at least one animal, wherein the one or more time-domain feature learning and extraction methods based upon at least one of: convolutional dictionary learning (CDL), convolutional sparse coding, translation invariant dictionary learning, cycle-by-cycle analysis, phase estimation or Hilbert transform methods, sliding window matching, template matching, empirical mode decomposition, recurrent neural networks or time delay neural networks or convolutional neural networks trained on features or short segments, dynamic time warping, motif learning or discovery, adaptive time-domain signal processing, temporal representation learning, or one or more unsupervised or semi-supervised machine learning algorithms.

In another aspect, the extrema and time-domain features may include a plurality of peaks and troughs between rising and falling zero-crossings of the beta wave events, wherein the at least one of the extrema includes a plurality of central troughs detected from the beta wave events.

Further, the processor of the first computing device may be configured to execute the instructions to display the mean waveforms aligned by at least one of the extrema to inform predictive biomarkers and targeted interventions.

According to other aspects, the present disclosure relates to a computer-implemented method configured to detect and analyze non-stationary, transient, or locally structured signals from longer time-series data of obtained beta waveforms to characterize a biomarker of a brain state or condition. An example method may comprise obtaining, by a processor of a first computing device, recordings of electrical activity arising from a brain of at least one animal; detecting, via the processor, beta wave events from the recordings by at least extracting extrema and time-domain features in unnormalized representations of the recordings; processing, via the processor, the beta wave events to generate a first representation of short-time segments representing a plurality of amplitude fluctuations indicating the extrema and a second representation for identifying temporal positions of the extrema in the first representation; comparing, via the processor, statistical distributions of a plurality of feature characteristics of the beta wave events based upon extracted extrema and time-domain features and the first and second representations; and generating, via the processor, mean waveforms of signals from at least one condition aligned by the temporal positions assigned to at least one of extracted feature types to indicate a brain state of the at least one animal. In one aspect, the extrema and time-domain features may comprise characteristics selected from the group consisting of: a peak time, a trough time, a peak amplitude, a trough amplitude, an oscillation period, a peak width, a trough width, an inter-peak timing, and an inter-trough timing.

In some implementations, the method may further comprise collecting, by a second computing device, the recordings of the at least one animal via a plurality of sensors, wherein the recordings comprise at least one of magneto- or electroencephalograph (M/EEG) recordings, local field potential (LFP) recordings, or electrocorticogram (ECoG) recordings; determining, via the processor, empirical distributions of the characteristics; and simulating, via resampling, an effect of alterations of empirical distributions of at least one of the characteristics on the mean waveforms.

In another embodiment, the method may comprise storing, via the processor, normative data of waveform features of the beta wave events and empirical or probability distributions or likelihoods thereof; and employing, via the processor, one or more time-domain feature learning and extraction methods to perform prediction of clinical indication and functional brain states of the at least one animal, wherein the one or more time-domain feature learning and extraction methods based upon at least one of: CDL, convolutional sparse coding, translation invariant dictionary learning, cycle-by-cycle analysis, phase estimation or Hilbert transform methods, sliding window matching, template matching, empirical mode decomposition, recurrent neural networks or time delay neural networks or convolutional neural networks trained on features or short segments, dynamic time warping, motif learning or discovery, adaptive time-domain signal processing, temporal representation learning, or one or more unsupervised or semi-supervised machine learning algorithms.

Moreover, the extrema and time-domain features may include a plurality of peaks and troughs between rising and falling zero-crossings of the beta wave events, wherein the at least one of the extrema includes a plurality of central troughs detected from the beta wave events. The method may also comprise displaying, via the processor, the mean waveforms aligned by at least one of the extrema to inform predictive biomarkers and targeted interventions.

In accordance with additional aspects, the present disclosure relates to a non-transitory machine-readable medium storing machine executable instructions for a computing server system that is configured to detect and analyze non-stationary, transient, or locally structured signals from longer time-series data of obtained beta waveforms to characterize a biomarker of a brain state or condition. For example, the machine executable instructions may be configured for: obtaining, by a processor of a first computing device, recordings of electrical activity arising from a brain of at least one animal; detecting, via the processor, beta wave events from the recordings by at least extracting extrema and time-domain features in unnormalized representations of the recordings; processing, via the processor, the beta wave events to generate a first representation of short-time segments representing a plurality of amplitude fluctuations indicating the extrema and a second representation for identifying temporal positions of the extrema in the first representation; comparing, via the processor, statistical distributions of a plurality of feature characteristics of the beta wave events based upon extracted extrema and time-domain features and the first and second representations; and generating, via the processor, mean waveforms of signals from at least one condition aligned by the temporal positions assigned to at least one of extracted feature types to indicate a brain state of the at least one animal. In one aspect, the extrema and time-domain features may comprise characteristics selected from the group consisting of: a peak time, a trough time, a peak amplitude, a trough amplitude, an oscillation period, a peak width, a trough width, an inter-peak timing, and an inter-trough timing.

Furthermore, according to one embodiment, the non-transitory machine-readable medium may comprise instructions for collecting, by a second computing device, the recordings of the at least one animal via a plurality of sensors, wherein the recordings comprise at least one of magneto- or electroencephalograph (M/EEG) recordings, local field potential (LFP) recordings, or electrocorticogram (ECoG) recordings; determining, via the processor, empirical distributions of the characteristics; simulating, via resampling, an effect of alterations of the empirical distributions of at least one of the characteristics on the mean waveforms; and displaying, via the processor, the mean waveforms aligned by at least one of the extrema to inform predictive biomarkers and targeted interventions.

In further embodiments, the non-transitory machine-readable medium may comprise instructions for storing normative data of waveform features of the beta wave events and empirical or probability distributions or likelihoods thereof; and employing, via the processor, one or more time-domain feature learning and extraction methods to perform prediction of clinical indication and functional brain states of the at least one animal, wherein the one or more time-domain feature learning and extraction methods based upon at least one of: CDL, convolutional sparse coding, translation invariant dictionary learning, cycle-by-cycle analysis, phase estimation or Hilbert transform methods, sliding window matching, template matching, empirical mode decomposition, recurrent neural networks or time delay neural networks or convolutional neural networks trained on features or short segments, dynamic time warping, motif learning or discovery, adaptive time-domain signal processing, temporal representation learning, or one or more unsupervised or semi-supervised machine learning algorithms.

In some embodiments, the extrema and time-domain features may include a plurality of peaks and troughs between rising and falling zero-crossings of the beta wave events, wherein the at least one of the extrema includes a plurality of central troughs detected from the beta wave events.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplary pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 illustrates a diagram of a computing system for dissecting M/EEG beta waveform features to reveal predictive biomarkers of neuronal mechanisms at a cellular and circuit level, according to an exemplary aspect;

FIG. 2 illustrates a block diagram of various components and modules of the system of FIG. 1, according to an exemplary aspect;

FIG. 3 illustrates example averaged beta event waveforms having stereotyped triphasic shapes with a central trough, according to an exemplary aspect;

FIG. 4 illustrates a flowchart of an example waveform learning and feature extraction protocol used by the system of FIG. 1, according to an exemplary aspect;

FIG. 5(A) illustrates a first temporal representation of atom #0 in a tactile detection dataset, according to an exemplary aspect;

FIG. 5(B) illustrates a second temporal representation of atom #1 in a tactile detection dataset, according to an exemplary aspect;

FIG. 5(C) illustrates a third temporal representation of atom #2 in a tactile detection dataset, according to an exemplary aspect;

FIG. 5(D) illustrates a fourth temporal representation of atom #3 in a tactile detection dataset, according to an exemplary aspect;

FIG. 6(A) illustrates an example sparse code for a selected trial, according to an exemplary aspect;

FIG. 6(B) illustrates original and reconstructed signals for the selected trial of FIG. 6(A), according to an exemplary aspect;

FIG. 7 illustrates an example raster of learned atoms within beta event span across trials, according to an exemplary aspect;

FIG. 8 illustrates peak and trough positions identified by using the atoms' extrema and locations in the sparse code of FIG. 6(A), according to an exemplary aspect;

FIG. 9 illustrates an example raster of detected peaks and troughs within beta event span across trials, according to an exemplary aspect;

FIG. 10(A) illustrates mean resting-state beta waveforms in postcentral gyrus of a plurality of subjects aligned by detected central troughs, according to an exemplary aspect;

FIG. 10(B) illustrates mean resting-state beta waveforms in caudal anterior cingulate of a plurality of subjects aligned by detected central troughs, according to an exemplary aspect;

FIG. 10(C) illustrates mean resting-state beta waveforms in precuneus of a plurality of subjects aligned by detected central troughs, according to an exemplary aspect;

FIG. 11 illustrates mean waveforms aligned by troughs detected from group-level, sub-cycle in detected and non-detected sensory trials, according to an exemplary aspect;

FIG. 12 illustrates mean waveforms aligned by central troughs detected from patients who convert or not convert to AD, according to an exemplary aspect;

FIG. 13 illustrates a table of features extracted from a given feature extraction technique and their respective characteristics, including amplitude and timing relative to a central trough, according to an exemplary aspect;

FIG. 14 illustrates empirical distributions of amplitudes of peak features categorized by their order relative to a central trough demonstrating a strong statistical difference between representative young and old subjects, according to an exemplary aspect;

FIG. 15 illustrates empirical distributions of amplitudes of trough features categorized by their order relative to a central trough demonstrating a weak statistical difference between representative young and old subjects, according to an exemplary aspect;

FIGS. 16 and 17 respectively illustrate empirical distributions of trough and peak timing features relative to a central trough and categorized by their order relative to the central trough demonstrating varying degrees of statistical differences between representative young and old subjects, according to an exemplary aspect; and

FIG. 18 illustrates a flowchart of a feature learning and extraction computer-implemented method for detecting and analyzing non-stationary, transient, or locally structured signals from longer time-series data of obtained beta waveforms of an animal to characterize a biomarker of a brain state or condition, according to an exemplary aspect.

### DETAILED DESCRIPTION

Various aspects of the present disclosure will be described with reference to the drawings, wherein like reference numerals are used to refer to like elements throughout. In the following description, for purposes of explanation, numerous specific details are set forth in order to promote a thorough understanding of one or more aspects of the present disclosure. It may be evident in some or all instances, however, that any aspects described below can be practiced without adopting the specific design details described below.

Among other things, the present disclosure may relate to a system, method, and apparatus for novel biomarker discovery using electromagnetic beta signal waveforms for prediction and diagnosis of neurological disorders and brain states. In one aspect, the system, method, and apparatus disclosed herein may be configured to examine certain time-domain characteristics of M/EEG beta waveforms across multiple tasks and datasets, including tactile detection, aging, and AD, PD and dementia. The disclosed aspects may be utilized for providing personalized therapy to caregivers of patients with dementia.

Age-related changes in stereotypical waveform features of both beta oscillations and evoked waveforms persist across subjects and reflect systematic alterations in inhibition, synchrony, and connectivity of pyramidal neurons within canonical circuits of the neocortex. In accordance with aspects of the present disclosure, two types of M/EEG signals may be obtained and analyzed: beta oscillations and auditory-evoked mismatch negativity. Beta transient oscillations (e.g., high 15-29 Hz power components of the M/EEG signals) are involved in cortical processing and occur when a subject is at rest. A beta waveform may be typically characterized by its average across many observations, but is shaped by statistical variations (of peak/trough timing, size, and shape) at the level of individual observations. The beta signal has utility as a biomarker due to its high-amplitude characteristics, its mechanistic links to activity in the neocortical column and thalamocortical network, and its vulnerability to clinical and functional states. Existing methods, however, do not characterize variations of beta waveforms at the level of individual observations.

On the other hand, mismatch negativity (MMN) is an evoked response thought to index disruptions in adaptive early sensory processing. Like the resting-state beta, MMN is generated by pyramidal neurons within early sensory circuits adapting to specific stimuli. While mechanisms underlying beta and MMN signals have been studied using rodent recordings and biophysical modeling, the consequences of aging-related processes on these mechanisms is unknown.

Systematic changes in the two types of M/EEG signals have been found to occur with healthy aging. However, precise waveform features of each signal remain underexplored, leading to missed opportunities to relate these signals to the dynamics of underlying neural ensembles. In recognizing that the waveform structure of resting-state beta and MMN signals is shaped in common by known aging-related processes affecting circuit mechanisms in Layer 2/3 and Layer 5 pyramidal cells, the present disclosure leverages M/EEG datasets and simulation-based neural modeling to examine the age-related alternations in waveform shapes, and to uncover cellular and circuit neurophysiology underlying these changes.

In some aspects, the present disclosure identifies that beta events localized to the somatosensory cortex may exhibit similar morphological deformations across tasks and populations that suggest common changes with aging and disease that relate to decline in somatosensory perception. The standard beta waveform, including an alternating series of peaks and troughs, varies across cycles in instantaneous frequency and amplitude. When aligning to the trough of the largest amplitude, consistent differences in beta waves may be identified in the shape and size of surrounding peaks in pre-stimulus non-detected trials, older age cohorts, and patients who later convert to dementia. This consistency may indicate a common underlying mechanism that influences beta generation, which may reflect a brief temporal pattern of synaptic excitatory drive at distal pyramidal dendrites lasting ˜50 ms. Among other features, the present disclosure relates to the functional role of beta oscillations in cognition and brain states, and the use of a generalizable beta waveform signature to inform predictive biomarkers and targeted interventions.

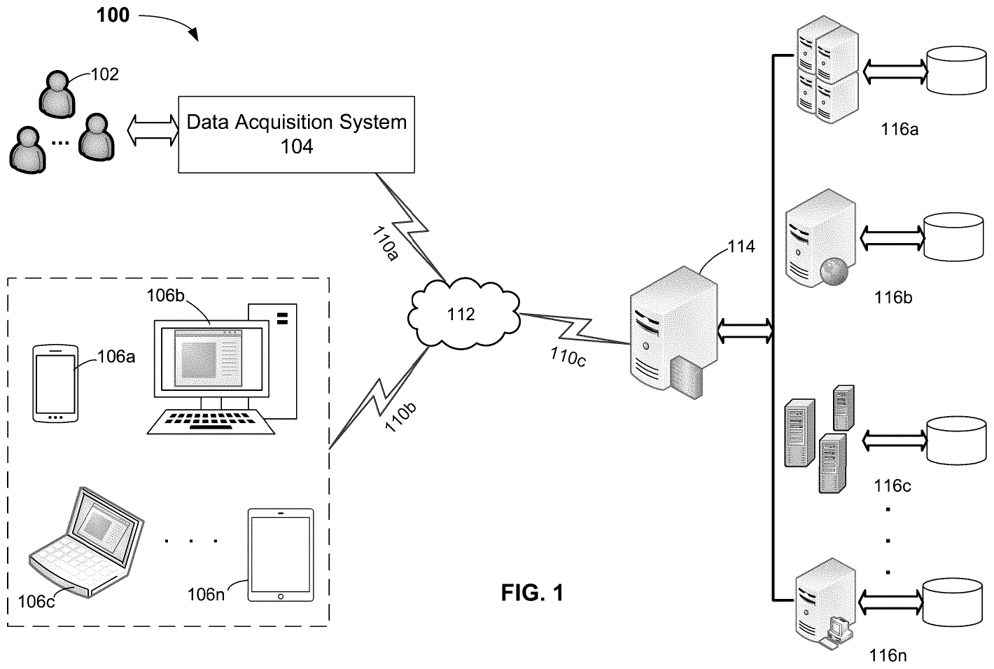

Referring to FIG. 1, in accordance with aspects, a computing system 100 deployed within a server-based computing environment and communication network may be configured to obtain recordings or data of electrical activity arising from individuals 102's brain via a data acquisition system 104, detect and extract a number of selected features (e.g., extrema and time-domain features) within selected waveforms (e.g., beta wave events) in the obtained recordings using at least one computing device 106a, 106b, 106c . . . 106n and/or a backend computing server 114, determine statistical distributions and variations of the detected extrema and time-domain features, and use statistical distributions and variations of these features to predict clinical indications or functional brain states associated with the obtained recordings. The recordings may comprise at least one of M/EEG recordings, LFP recordings, or ECOG recordings. It should be appreciated that the individual 102 may be an animal (e.g., a rodent, a cat, a monkey, a dog, a cow, or any mammals).

In one example study, the data acquisition system 104 of the present disclosure may be configured to obtain M/EEG recordings from a group of healthy aging individuals 102. A cross-sectional dataset may target 700 participants with 50 men and 50 women from each age decade. M/EEG data may be collected using a 306-channel VectorView MEG system, which includes 102 magnetometers and 204 orthogonal planar gradiometers. The M/EEG data may be sampled at 1 KHz with a high-pass filter of 0.03 Hz. Vertical and horizontal electrooculogram (VEOG, HEOG) signals may be recorded to monitor blinks and eye movements, along with an electrocardiogram (ECG) signal. For each participant, there may be several data collection sessions (e.g., 3 separate sessions). During the MEG resting state recording, participants had their eyes closed for a selected period of time (e.g., at least 8 minutes and 40 seconds).

In another aspect, the data acquisition system 104 may be configured to collect TI-weighted magnetic resonance imaging (MRI) datasets from certain individuals 102 using a 3 Tesla total imaging matrix (TIM) Siemens Trio scanner with a 32-channel head coil in a single 1-hour session. Participants lacking MRI data may not be included in subsequent analyses.

In another example study, the data acquisition system 104 of the present disclosure may be configured to obtain M/EEG recordings from a group of individuals 102 having mild cognitive impairment (MCI). In particular, 84 people may be subdivided into healthy controls, subjects with MCI who convert to AD within 2.5 years, and subjects with MCI who do not convert. M/EEG data may be collected using a 306-channel VectorView MEG system comprised of 102 magnetometers and 204 gradiometers, positioned within a magnetically shielded room. Collected data may be sampled at a rate of 1000 Hz, with an online anti-alias band-pass filter ranging from 0.1 to 330 Hz. Additionally, individual T1-weighted MRI scans of each participant may be captured using a 1.5 T General Electric MRI scanner equipped with a high-resolution antenna and a homogenization PURE filter. In one embodiment, MRI parameters may include a fast spoiled gradient echo sequence with repetition time/echo time/inversion time values of 11.2/4.2/450 ms; a flip angle of 12°; a slice thickness of 1 mm; a 256×256 matrix; and a field of view of 256 mm. For each participant, there may be several data collection sessions (e.g., 3 separate sessions). During the resting-state recording, participants had their eyes closed for a selected period of time (e.g., at least 5 minutes).

In yet another example study, the data acquisition system 104 of the present disclosure may be configured to perform tactile detection and obtain M/EEG recordings of a study population 102. For example, 7 neurologically healthy, right-handed 18-45 year olds may participate a tactile detection task. Their M/EEG data may be collected using a 306-channel VectorView magnetoencephalography comprising 306 sensors arranged in triplets: two planar gradiometers and a magnetometer at 102 sites. Collected data may be sampled at 600 Hz, with a band-pass of 0.01-200 Hz initially, later re-averaged off-line using a bandpass of 0.03-200 Hz. Epochs with electrooculograms (EOG) peak-to-peak amplitude exceeding 150 μV may be excluded from further analysis. Vertical and horizontal EOG may be monitored using electrodes placed near the left eye of each participant. Subjects may experience brief taps on their right hands, delivered as a single cycle of 100 Hz sine wave (10 ms duration) via a custom piezoelectric device. Individual detection thresholds may be pre-determined using a convergence procedure. During the MEG imaging, 70% of trials may have stimuli at the perceptual threshold, with stimulus strength adjusted dynamically based on subject responses. Trial duration may be set to 3 seconds, with a 60 dB, 2 kHz auditory cue indicating trial onset. During this auditory cue, the finger tap stimulus may be presented between 500 and 1500 ms in 100 ms intervals. Subjects reported stimulus detection or non-detection using button presses with their left hands. During subsequent data collection sessions, each subject may have eight runs, each consisting of 120 trials. Within these trials, suprathreshold stimuli (10% of all trials) and null trials (20%) may be interleaved randomly with threshold stimuli. Threshold stimuli are of enough energy or potential to produce an action potential (nerve impulse). Suprathreshold stimuli also produce an action potential, but their strength is higher than the threshold stimuli.

In one aspect, the data acquisition system 104 may be configured to transmit the M/EEG signals of the individuals 102 to a selected computing device or system (e.g., one of 106a, 106b, 106c . . . 106n) and/or a backend computing server 114 via a network 112 and communication protocols 110a, 110b, 110c.

According to one embodiment, an application, which may be a mobile or web-based application (e.g., native iOS or Android Apps), may be downloaded and installed on the selected computing device or system 106a, 106b, 106c, . . . 106n for instantiating various modules for processing and analyzing the M/EEG data, and interacting with a user of the application, among other features. For example, such an application may be used by caregivers of each individual 102, medical professionals, neurological disorders researchers, M/EEG technicians or technologists, trained staff (e.g., nurses, support staff, monitor technicians), and other end-users. Automated agents, scripts, playback software, and the like acting on behalf of one or more people may also be users. Such a user-facing application of the system 100 may include a plurality of modules executed and controlled by the processor of the hosting computing device or system 106a, 106b, 106c, . . . 106n for processing M/EEG signals received from the data acquisition system 104 using various algorithms, as will be described fully below. Computing device 106a, 106b, 106c, . . . 106n hosting the mobile or web-based application may be configured to connect, using a suitable communication protocols 110b, 110c and network 112, to the backend computing server 114. Here, communication network 112 may generally include a geographically distributed collection of computing devices or data points interconnected by communication links and segments for transporting signals and data therebetween. Communication protocol(s) 110a, 110b, 110c may generally include a set of rules defining how computing devices and networks may interact with each other, such as frame relay, Internet protocol (IP), transmission control protocol (TCP), user datagram protocol (UDP), hypertext transfer protocol (HTTP). It should be appreciated that the system 100 of the present disclosure may use any suitable communication network, ranging from local area networks (LANs), wide area networks (WANs), cellular networks, to overlay networks and software-defined networks (SDNs), a packet data network (e.g., the Internet), mobile telephone networks (e.g., cellular networks, such as 4G or 5G), Plain Old Telephone (POTS) networks, and wireless data networks (e.g., Institute of Electrical and Electronics Engineers (IEEE) 802.11 family of standards known as Wi-Fi®, WiGig®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, virtual private networks (VPN), Bluetooth, Near Field Communication (NFC), or any other suitable network.

In some embodiments, the backend computing server 114 may be Cloud-based or an on-site server. The term “server” generally refers to a computing device or system, including processing hardware and process space(s), an associated storage medium such as a memory device or database, and, in some instances, at least one database application as is well known in the art. The backend computing server system 114 may provide functionalities for any connected devices such as sharing data or provisioning resources among multiple client devices or performing computations for each connected client device. According to one embodiment, within a Cloud-based computing architecture, the backend computing server 114 may provide various Cloud computing services using shared resources. Cloud computing may generally include Internet-based computing in which computing resources are dynamically provisioned and allocated to each connected computing device or other devices on-demand, from a collection of resources available via the network or the Cloud. Cloud computing resources may include any type of resource, such as computing, storage, and networking. For instance, resources may include service devices (firewalls, deep packet inspectors, traffic monitors, load balancers, etc.), computing/processing devices (servers, central processing units (CPUs), graphics processing units (GPUs), random access memory, caches, etc.), and storage devices (e.g., network attached storages, storage area network devices, hard disk drives, solid-state devices, etc.). In addition, such resources may be used to support virtual networks, virtual machines, databases, applications, etc. The term “database,” as used herein, may refer to a database (e.g., relational database management system (RDBMS) or structured query language (SQL) database), or may refer to any other data structure, such as, for example a comma separated values (CSV), tab-separated values (TSV), JavaScript Object Notation (JSON), extendible markup language (XML), TEXT (TXT) file, flat file, spreadsheet file, and/or any other widely used or proprietary format. In some embodiments, one or more of the databases or data sources may be implemented using one of relational databases, flat file databases, entity-relationship databases, object-oriented databases, hierarchical databases, network databases, NoSQL databases, and/or record-based databases.

Cloud computing resources accessible using any suitable communication network (e.g., Internet) may include a private Cloud, a public Cloud, and/or a hybrid Cloud. Here, a private Cloud may be a Cloud infrastructure operated by an enterprise for use by the enterprise, while a public Cloud may refer to a Cloud infrastructure that provides services and resources over a network for public use. In a hybrid Cloud computing environment which uses a mix of on-premises, private Cloud and third-party, public Cloud services with orchestration between the two platforms, data and applications may move between private and public Clouds for greater flexibility and more deployment options. Some example public Cloud service providers may include Amazon (e.g., Amazon Web Services® (AWS)), IBM (e.g., IBM Cloud), Google (e.g., Google Cloud Platform), and Microsoft (e.g., Microsoft Azure®). These providers provide Cloud services using computing and storage infrastructures at their respective data centers and access thereto is generally available via the Internet. Some Cloud service providers (e.g., Amazon AWS Direct Connect, Microsoft Azure ExpressRoute) may offer direct connect services and such connections typically require users to purchase or lease a private connection to a peering point offered by these Cloud providers.

In certain implementations, the backend computing server 114 (e.g., Cloud-based or an on-site server) of the present disclosure may be configured to connect with various data sources or services 116a, 116b, 116c, . . . 116n. As will be described fully below, the backend computing server system 114 may be configured to host, train, operate, and/or incorporate any suitable type of learning model (e.g., at least one of 116a, 116b, 116c, . . . 116n) for determining time-domain characteristics of beta waveforms across multiple tasks and datasets, including tactile detection, aging, and PD, AD or dementia based at least upon M/EEG signals obtained from individuals 102.

In some embodiments, the learning model employed in the present disclosure may be based on selected time-domain feature learning and extraction methods including but not limited to: CDL, convolutional sparse coding, translation invariant dictionary learning, cycle-by-cycle analysis, phase estimation or Hilbert transform methods, sliding window matching, template matching, empirical mode decomposition, recurrent neural networks or time delay neural networks or convolutional neural networks trained on features or short segments, dynamic time warping, motif learning or discovery, adaptive time-domain signal processing, temporal representation learning, or one or more unsupervised or semi-supervised machine learning algorithms. In one aspect, at least one of the time-domain feature learning and extraction methods may be used to learn or extract non-stationary, transient, or locally structured signals from longer time-series data, thereby locating, characterizing, analyzing, and comparing a plurality of features (e.g., peaks, troughs, extrema, non-stationarities, etc.) within the obtained beta waveform or event-related potential (ERP).

In one implementation, a computing model may use multivariate convolutional sparse coding (CSC), which is a specification of the broader class of CDL algorithms. As used herein, the disclosed CDL may represent the multivariate neural signals as a set of spatiotemporal patterns (atoms), with their respective onset times and magnitudes (activations).

According to further embodiments, the backend computing server system 114 may be configured to host, train, operate, and/or incorporate any suitable machine learning models (e.g., at least one of 116a, 116b, 116c, . . . 116n) for real-time analysis of brain states using the M/EEG recordings obtained from the data acquisition system 104 and/or computing devices 106a, 106b, 106c, . . . 106n. Example machine learning algorithms may include but not limited to random forest, Boosting, Naïve Bayesian Classifier, k-Nearest Neighbors (KNN) and Support Vector Machine (SVM). According to one implementation, a random forest classifier may be used to enable prediction of clinical indications and functional brain states. Random forest classifier is a collection or ensemble of classification and regression trees trained on datasets of the same size as training set, called bootstraps, created from a random resampling on the training set itself. Once a tree is constructed, a set of bootstraps, which do not include any particular record from the original dataset (out-of-bag (OOB) samples), is used as test set. The error rate of the classification of all the test sets is the OOB estimate of the generalization error. For the bagged classifiers, the OOB error is accurate as using a test set of the same size as the training set. Thus, using the OOB estimate may remove the need for a separate test set. To classify new input data, each individual classification and regression tree votes for one class and the forest predicts the class that obtains the plurality of votes. Hyperparameters (e.g., the number of trees in the forest, the maximum depth of each tree, etc.) are arguments passed to the constructor of the random forest classifier class. In one embodiment, the computing server system 114 may determine and fine tune the hyperparameter of the random forest classifier to improve its performance. Generally, the performance of a training model may be evaluated by confusion matrix, precision, recall, fl score, accuracy, and the probability of each prediction.

In accordance with important aspects, the present disclosure relates to applying a CDL-based waveform characterization methodology to individual beta cycles in magnetoencephalographic (MEG) sources computed with a surface-normal orientation constraint to extract temporally localized time-domain features representing a beta waveform shape. The extracted features may be analyzed and compared across various experimental conditions and subject groups.

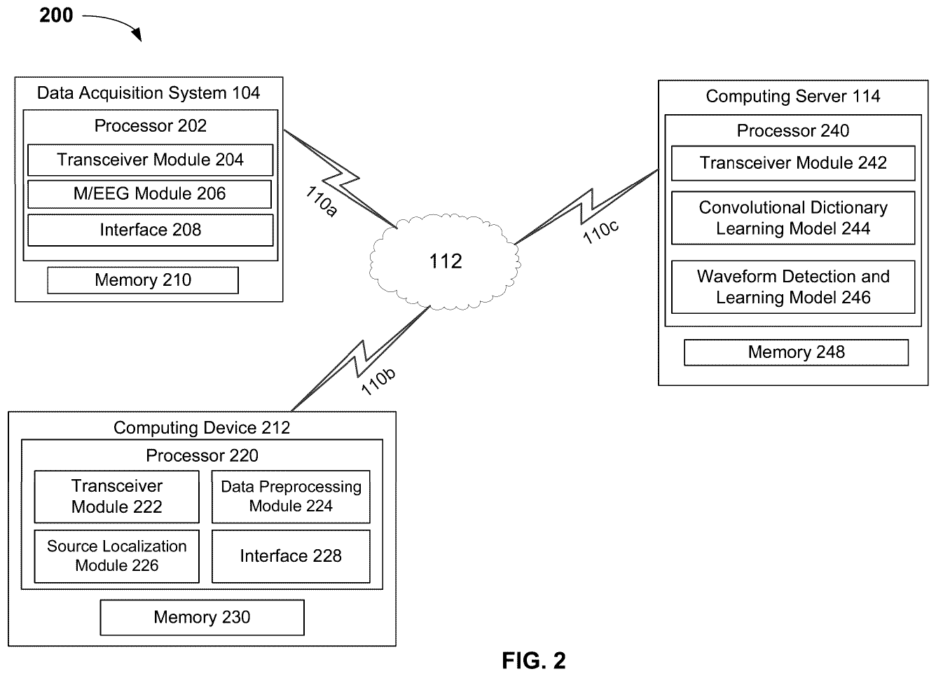

FIG. 2 is a block diagram 200 illustrating various components and modules of the data acquisition system 104, a computing device 212 (e.g., one of computing device 106a, 106b, 106c . . . 106n), and the backend computing server 114, in accordance with aspects of the present disclosure.

As described above with respect to FIG. 1, the data acquisition system 104 may include any suitable computing system for collecting M/EEG recordings from individuals 102. Such a computing system may generally include at least one microcontroller or processor 202 configured to control and execute a plurality of modules such as a transceiver module 204, a M/EEG module 206, and an interface 208. The term “module” as used herein refers to a real-world device, component, or arrangement of components and circuitries implemented using hardware, such as by an application specific integrated circuit (ASIC) or field-programmable gate array (FPGA), for example, or as a combination of hardware and software, such as by e.g., the processor 202 and a set of instructions to implement the module's functionality, which (while being executed) transform the microcontroller into a special purpose device. A module may also be implemented as a combination of the two, with certain functions facilitated by hardware alone, and other functions facilitated by a combination of hardware and software. Each module may be realized in a variety of suitable configurations, and should not be limited to any example implementation exemplified herein. For example, the transceiver module 204 may be configured by the processor 202 to exchange various information and data with other modules and/or computing devices connected with the data acquisition system 104. The M/EEG module 206 may include an array of sensors, magnetometers, gradiometers, MRI scanner, any other suitable computing systems and devices configured to obtain M/EEG recordings from individuals 102. In some embodiments, the M/EEG module 206 may be controlled by the processor 202 to preprocess the M/EEG recordings to generate obtain at least the temporal, frequency, and/or phase features of these signals. Interface 208 may be configured to allow a user to control and access different modules and computing devices connected with the data acquisition system 104. Information relating to the control and the processing of the obtained M/EEG recordings may be presented to a user via the interface 208 which may include any suitable graphical user interface, command line interface, menu-driven user interface, touch user interface, voice user interface, form-based user interface, natural language user interface, and mobile user interface (e.g., graphical and touch-sensitive display features associated with mobile computing devices such as smartphones and tables).

Memory 210, which is coupled to processor 202, may be configured to store at least a portion of information obtained by the data acquisition system 104 (e.g., M/EEG recordings obtained from individuals 102). In one aspect, memory 210 may be a non-transitory machine-readable medium configured to store at least one set of data structures or instructions (e.g., software) embodying or utilized by at least one of the techniques or functions described herein. It should be appreciated that the term “non-transitory machine-readable medium” may include a single medium or multiple media (e.g., one or more caches) configured to store at least one instruction. The term “machine-readable medium” may include any medium that is capable of storing, encoding, or carrying instructions for execution by all modules of the data acquisition system 104 and that cause these modules to perform at least one of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks.

Subsequently, the data acquisition system 104 may transmit the obtained M/EEG recordings, via the transceiver module 204, to a selected computing device 212 (e.g., one of computing device 106a, 106b, 106c . . . 106n in FIG. 1). According to one implementation, the computing device 212 may include a processor 220 configured to control and execute a plurality of modules including but not limited to a transceiver module 222, a data preprocessing module 224, a source localization module 226, and an interface 228. Memory 230, which is coupled to the processor 220, may be configured to store at least a portion of information obtained by the computing device 212 and store at least one set of data structures or instructions (e.g., software) embodying or utilized by at least one of the techniques or functions described herein.

In one embodiment, the transceiver module 222 may be controlled by the processor 220 to exchange various information and data with other modules and/or computing devices deployed within the system 100 and connected with the computing device 212. Specifically, M/EEG recordings received from the data acquisition system 104 may be preprocessed with uni- and multi-variate techniques by a data preprocessing module 224. For example, the measured univariate time-series of each electric or magnetic recording channel may be represented as a row vector, and may be processed via any appropriate techniques including but not limited to mean subtraction, band-pass, high-pass and/or low-pass filtering, detrending, harmonic analysis, and/or down-sampling. Thereafter, signals may be transformed into time-frequency/scale domains with Fourier or wavelet methods (e.g., Morlet wavelet decomposition or other suitable methods), or may be band-pass filtered and transformed to extract the instantaneous amplitude and phase. Frequency specific noise may be filtered out in the frequency or time domains. Filtering or time-frequency analysis may be used by the data preprocessing module 224 for identifying wavelike activity and oscillations at specific frequency bands. Although the lower limit and higher limit of these frequency bands may vary but they are generally respectively around: the slow oscillation (<1 Hz), delta (1-4 Hz), theta (5-8 Hz), alpha (8-12 Hz), mu (8-12 Hz and 18-25 Hz), spindles (˜14 Hz), beta (13-30 Hz), gamma (30-80 Hz), high gamma or omega (80-200 Hz), ripples (˜200 Hz), and sigma bursts (˜600 Hz). In one example, the raw time signals of the M/EEG recordings may be processed by the data preprocessing module 224 using a band-pass filter with frequencies ranging from 1 Hz to 90-200 Hz.

Accurate localization of functional brain activity holds promise to enable novel treatments and assistive technologies that are in critical need by the aging society. Since M/EEG recordings capture the electromagnetic fields produced by neuronal currents of the human brain, they provide a fast and direct index of neuronal activity. A source model may be used to approximate the current density in the human brain. Mapping the activity of known sources in the brain to the corresponding M/EEG signals is called the forward problem. This problem may be solved by giving a parametric representation of the sources and by modeling how the electromagnetic field propagates through the brain compartments. According to the distributed source model, the neural current is assumed to be a continuous vector field inside the brain volume. According to the equivalent current dipole (ECD) model, the primary source of these electromagnetic signals is the electrical currents flowing through the apical dendrites of pyramidal neurons in the cerebral cortex, and the whole brain activity underlying the M/EEG measurements occurs only in a small number of clusters of thousands of synchronously activated pyramidal cortical neurons. In this setting, each cluster is represented by a point source (i.e., ECD), and the overall primary current distribution is approximated by the superposition of a given number of ECDs. The ECD model is currently the standard approach in clinical applications of M/EEG measurements, such as the pre-surgical localization of epileptic spikes. In order to model the propagation of the electromagnetic field through the human brain, it is important to obtain information relating to the physical and geometrical properties of the head, which may be gathered from high resolution anatomical MRI scans. Discretization of the differential equations governing the electromagnetic fields may be carried out using boundary element methods (BEM) or finite element methods (FEM). Different tissues such as the scalp, skull, cerebrospinal fluid, and brain have different conductivity characteristics and therefore attenuate the current to a different extent. For example, BEM may be used to model homogeneous and isotropic conductivity, and FEM may be used to model inhomogeneous and anisotropic conductivity. As part of the source localization, an inverse problem infers the location of the generators of brain activity from M/EEG data. Source localization may provide a single, unique best reconstruction of neural activity from a given dataset.

According to one implementation, the source localization module 226 of the computing device 212 may be configured by the processor 220 to reconstruct each participant's MRI. The MRI reconstruction may facilitate the digitization of the cortical surface for source estimation and provide a transformation to an average brain which aids in spatial normalization and group statistical analysis. Next, the source localization module 226 may carry out a forward model construction to delineate how sources in the brain create observable M/EEG signals. Using MRI data and known MEG sensor positions, the forward solution may be formulated. This process may be improved by digitizing the cortical surface for source estimation and employing a boundary element model for more precise calculation of the forward solution.

The source localization module 226 may also determine a source space definition which identifies potential source locations by segmenting the brain volume. A BEM model may be established to represent the conductive boundaries (e.g., scalp, skull, and brain). These boundaries may influence MEG measurements.

Co-registration between structural head images and functional M/EEG data may be used for anatomically-informed M/EEG data analysis by the source localization module 226. In one example study, each participant's MRI data may be registered to the MEG data, focusing on the alignment of anatomical landmarks (fiducial points) and additional head points. Further, the MEG head digitization data may be coordinated with the MRI using a semi-automated process.

The source localization module 226 may be configured to formulate an inverse problem solution by estimating the neural current distribution that resulted in the observed MEG data. Time courses for each region of interest (ROI), such as the sensorimotor cortex, may be determined based on the center of mass of the ROI. As a result, a three-dimensional (3D) vector source estimate may be produced. This 3D vector estimate may be subsequently projected to the vector perpendicular to the cortical surface. This projection may enable the computation of the dipole, signifying the primary neural current direction for that ROI.

In some embodiments, the computing device 212 may be a thin client device/terminal/application deployed within the system 100 and may be configured to perform certain preliminary processing of raw M/EEG signals and information from various users. Thereafter, the processed data may be transmitted to e.g., the computing server 114 to apply CDL or other computationally intensive processing and analysis to extract temporally localized time-domain features of beta event waveform shape, as will be described fully below. In one embodiment, interface 228 may include an application programming interface (API) interface configured to make one or more API calls therethrough. On the other hand, the computing server 114 may include an API gateway device (not shown) configured to receive and process API calls from various connected computing devices deployed within the system 100 (e.g., an operating system, a library, a device driver, an API, an application program, software or other module). Such an API gateway device may specify one or more functions, methods, classes, objects, protocols, data structures, formats and/or other features of the computing server 114 that may be used by the mobile or web-based application of the computing device 212. For example, the API interface may define at least one calling convention that specifies how a function associated with the computing server 114 receives data and parameters from a requesting device/system and how the function returns a result to the requesting device/system. It should be appreciated that the computing server 114 may include additional functions, methods, classes, data structures, and/or other features that are not specified through the API interface and are not available to a requesting computing device.

In certain embodiments, the computing server 114 may include a processor 240 configured to control and execute a plurality of modules including but not limited to a transceiver module 242, a CDL model 244, and a waveform detection and learning module 246. For example, the computing server 114 may use the waveform detection and learning model 246 for time-domain feature learning and extraction using any suitable computing models and algorithms other than the CDL-based approach used by the CDL model 244. In some embodiments, the waveform detection and learning model 246 may use convolutional sparse coding, translation invariant dictionary learning, cycle-by-cycle analysis, phase estimation or Hilbert transform methods, sliding window matching, template matching, empirical mode decomposition, recurrent neural networks or time delay neural networks or convolutional neural networks trained on features or short segments, dynamic time warping, motif learning or discovery, adaptive time-domain signal processing, temporal representation learning, or one or more unsupervised or semi-supervised machine learning algorithms. The waveform detection and learning model 246 may be configured to learn or extract non-stationary, transient, or locally structured signals from longer time-series data, thereby locating, characterizing, analyzing, and comparing a plurality of features (e.g., peaks, troughs, extrema, non-stationarities, etc.) within the obtained beta waveform or ERP.

Memory 248, which is coupled to the processor 240, may be configured to store at least a portion of information obtained by the computing server 114 and store at least one set of data structures or instructions (e.g., software) embodying or utilized by at least one of the techniques or functions described herein. In one embodiment, memory 248 may be configured to store normative data of beta waveform features across different clinical and functional brain states or processes for use by e.g., the CDL model 244, the waveform detection and learning model 246, or other suitable machine learning model connected with the computing server 114.

It should be appreciated that codes and instructions of certain modules of the computing server 114 (e.g., the CDL model 244 or the waveform detection and learning module 246) may be downloadable and stored on a non-transitory machine-readable medium of the computing device 212 (e.g., memory 230), such that the disclosed aspects in the present disclosure may be implemented locally on the computing device 212.

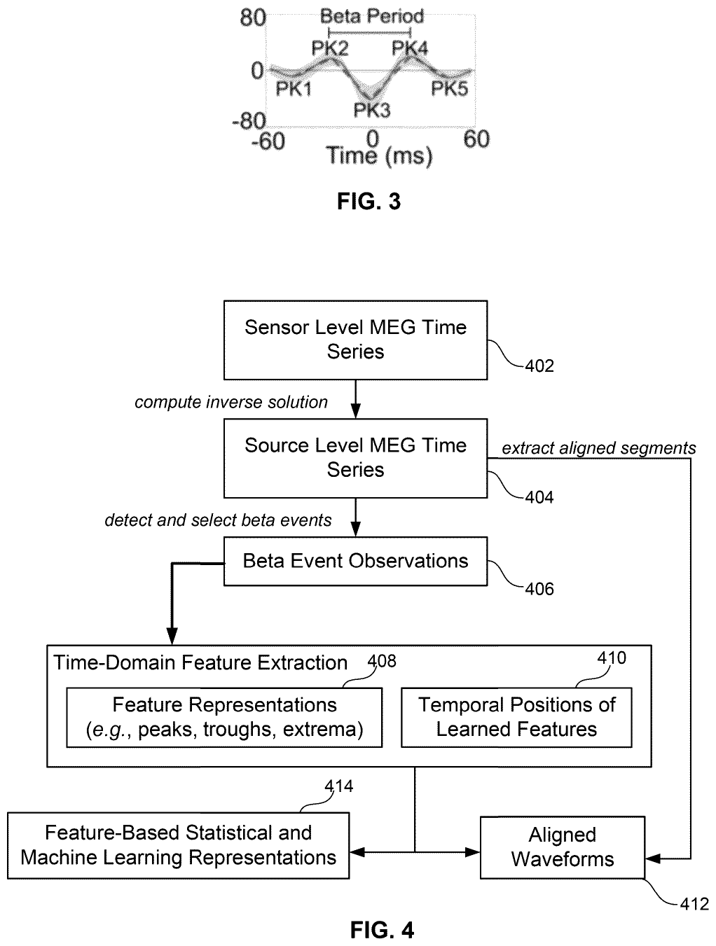

In accordance with one embodiment, the CDL model 244 or the waveform detection and learning module 246 may be configured to receive preprocessed M/EEG recordings from the computing device 212 and perform beta event detection. Spontaneous beta activity from somatosensory and frontal cortex emerged as non-continuous beta events typically lasting <150 ms with a stereotypical waveform. Neural oscillations often occur as a burst. A burst may be characterized by the number of oscillation cycles which it comprises. The waveform shape of a neural oscillation in time domain may be characterized by its rise-fall symmetry and peak-trough symmetry. The electrical currents underlying these signals show that beta events may emerge from the integration of synchronous bursts of excitatory synaptic drive targeting proximal and distal dendrites of pyramidal neurons, where the defining feature of a beta event was a strong distal drive that lasted one beta period (˜50 ms). As shown in FIG. 3, beta events may have a consistent temporal profile and averaged beta event waveforms have stereotyped triphasic shapes with a central trough (PK3). That is, high-power beta events have a stereotypical waveform where its polarity and temporal profile may be highly consistent within and across subjects and exhibited a statistically significant negative deflection. For waveform shape analysis, time points of rising and falling zero-crossings (i.e., voltage sign-changes) may be initially identified in the beta filtered signal (approximately between 13 and 30 Hz) using e.g., window-method based FIR filter. Next, beta oscillatory extrema (peaks and troughs) may be identified. Referring to FIG. 3, the maximum voltage (the “peak”) and minimum voltage (the “trough”) between zero-crossings may be indicated as PK1, PK2, PK3, PK4, and PK5. The time point of maximal voltage between a rising zero-crossing and a subsequent falling zero-crossing is defined as the peak. Similarly, the time point of minimal voltage between a falling zero-crossing and a subsequent rising zero-crossing is defined as a trough.

Extrema sharpness may be defined as the average difference between the voltage at the extrema and the voltages of a selected number of data points before and after the peak. Extrema sharpness increases as the absolute voltage difference between the extrema and the surrounding time points increases. Extrema width may be defined as the difference in time between the peak and trough of the average waveform.

In some embodiments, an average of a number of high-power beta events in somatosensory and frontal cortex from example subjects, aligned to the maximum extrema in the waveform closest to the time of greatest spectral power in the corresponding spectral beta event show that the waveform is not sinusoidal in nature. As shown in FIG. 3, a dominant trough (PK3) may be present across different events. Although the amplitude of the peaks in the aligned waveforms varies across subjects and brain areas, the temporal profiles are consistent and symmetric near the PK3 trough, indicating that a highly stereotypical underlying biophysical process may create this trough activity. Several key features defining the shape of the beta event waveforms may be statistically significant within and across subjects in both somatosensory and frontal cortex. For example, the amplitude of trough PK3 is negative, which is opposite in sign to the neighboring positive PK2 and PK4 peaks and has a greater absolute magnitude than that of the PK1 and PK5 peaks. The PK3 trough is also the sharpest peak in both somatosensory and frontal cortex (i.e., the slopes from PK2 to PK3 and from PK3 to PK4 are steeper than those between other peaks). Peaks and troughs other than PK3 trough may not exhibit consistent features across averaged beta waveforms. As such, the duration of the dominant PK3 trough may be used to determine the frequency of a specific beta event. PK3 duration is defined as the time between PK2 and PK4 extrema for each event. Further, the amplitude of PK3 trough may determine the power of the event.

In accordance with aspects of the present disclosure, the computing server 114 may be configured to detect statistically significant features of the M/EEG beta waveforms to identify major cyclic components (e.g., peaks and troughs) that explain the greatest variance in the data.

According to one embodiment, the CDL model 244 or the waveform detection and learning module 246 may analyze spontaneous signals from sensorimotor cortices in three different MEG datasets: somatosensory neocortex sources during pre-stimulus periods in tactile detection task; postcentral sources during eyes-closed resting state in healthy aging subjects (20-90 years old); and sensorimotor sources during eyes-closed resting-state in patients with MCI who later convert or do not convert to AD.

FIG. 4 illustrates a flowchart of an example waveform learning and feature extraction protocol used by the system 100 of FIG. 1. For example, the system 100 may be configured to compute the inverse solution of sensor level MEG time series 402 corresponding to the M/EEG recordings obtained from various individuals 102. The resulting source level MEG time series 404, which extract aligned segments within the beta waveforms, may be processed by the CDL model 244 or waveform detection and learning model 246 of the computing server 114 to detect and select beta events. In one aspect, the beta event observations 406 performed by the computing server 114 may extract time-domain features including feature representations 408 (e.g., peaks, troughs, extrema) and temporal positions of learned features 410. Thereafter, in one aspect, the computing server 114 may generate aligned waveforms 412 based on the outputs of the feature representations 408, the temporal positions of learned features 410, and the source level MEG time series 404. The computing server 114 may also generate feature-based statistical and machine learning representations based on the outputs of the time-domain features extraction 408 and 410.

In some aspects, the computing server 114 may detect beta transient events 406 based on received source level MEG time series 404. For example, spectral events may be initially identified by extracting all local maxima in the unnormalized (e.g., unaveraged) representation (TFR). For every ROI, transient high-power events may be characterized as local maxima that exceeded a threshold of six times the median within the beta frequency band (15-29 Hz). Every detected event may be output with an onset and offset time, among other attributes.

Subsequently, the computing server 114 may be configured to perform event sectioning. Specifically, within each identified event (between event onset and offset times), the field potential minimum may be located. One segment per event, consisting of 350 data samples forward and backward in time from the minimum, may be sectioned for subsequent steps.

According to one implementation, the computing server 114 may be configured to utilize Alpha-stable CDL, which is an unsupervised learning technique designed to capture the dynamic structure of non-stationary signals. For example, with respect to FIG. 4, the CDL model 244 may generate an atomic representation 408 of short-time segments representing large-amplitude fluctuations within the data as well as a sparse code 410 representing the temporal positions of learned atoms within the original data. Accordingly, peaks and troughs may be detected from beta waveforms across multiple contexts such as tactile detection, aging, and AD. As such, the CDL model 244 of the present disclosure may be used in analysis of beta events and waveform learning applications.

Additionally, temporal locations of troughs may be located in the time series using the minima of CDL-detected atoms and their sparse codes within the original time series. Raw beta event time series may be aligned by their trough times. Mean waveforms may be computed across time using the standard error of the mean across beta event samples in each cohort/condition. Mean waveforms and other waveform-related features may be used in predictive approaches in aging, disease, and functional-related applications.

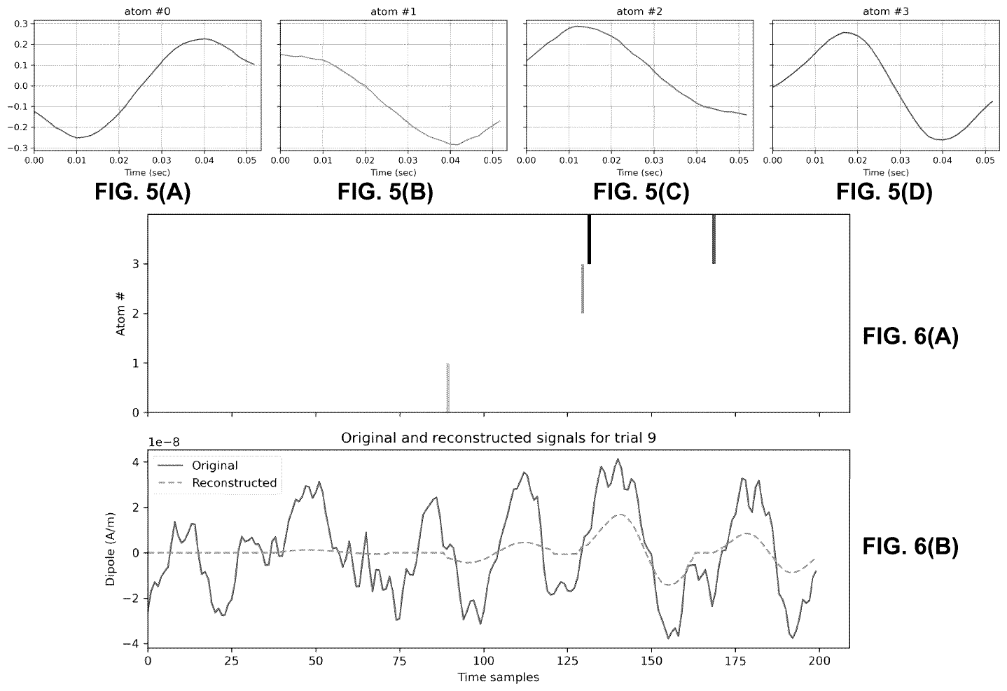

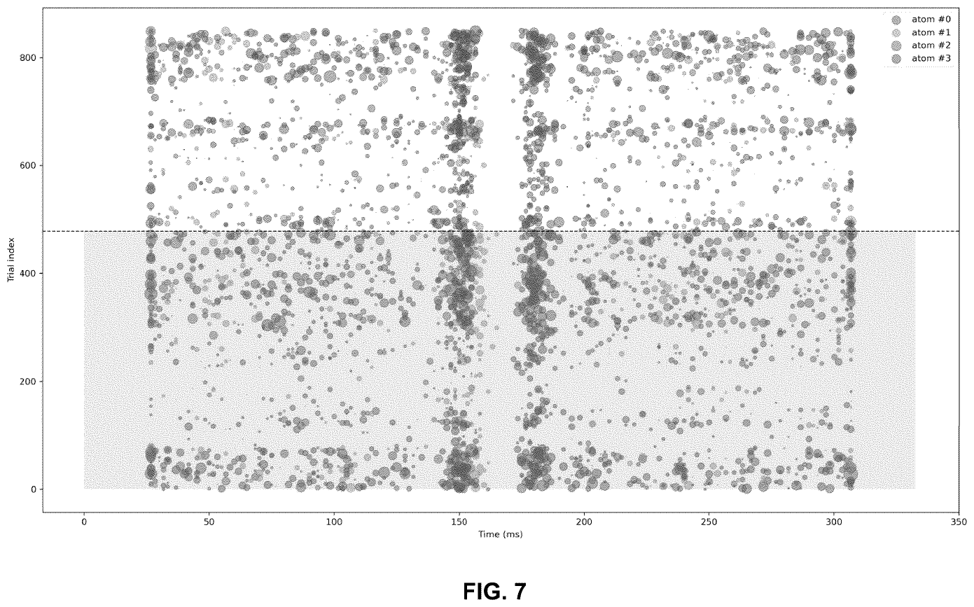

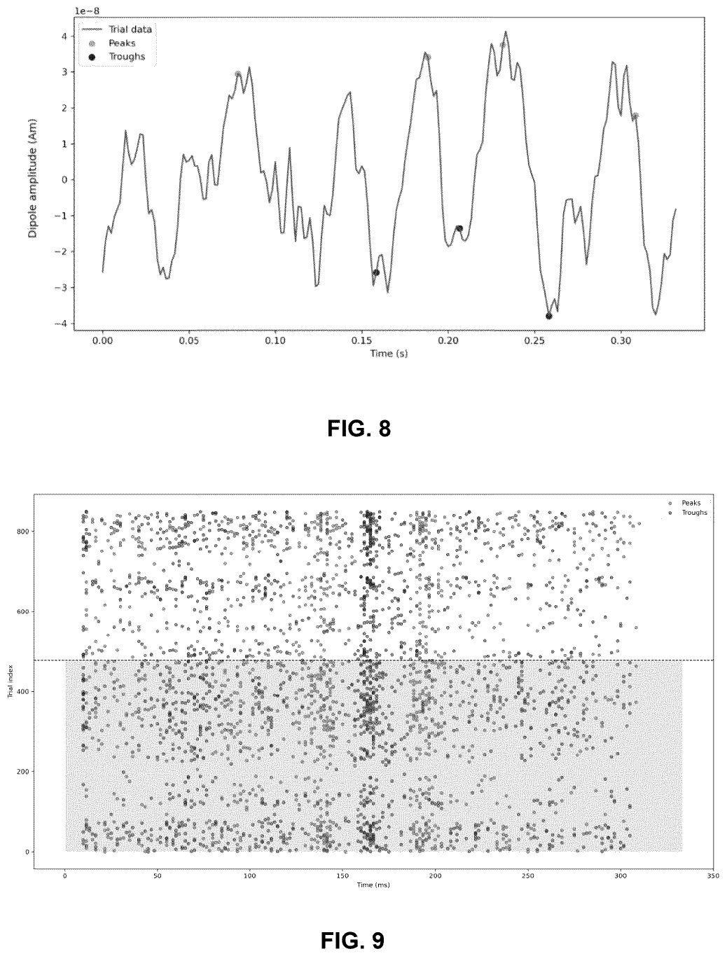

FIGS. 5(A) (atom #0), 5(B) (atom #1), 5(C) (atom #2), and 5(D) (atom #3) respectively illustrate a waveform learning procedure at group-level for a sub-cycle to identify basis components (i.e., atoms) comprising the M/EEG data. Specifically, four temporal representations of four atoms in tactile detection dataset are shown, wherein the atom length is 32 samples (˜ 53 ms). FIGS. 5(A)-5(D) collectively show CDL derived subcomponents explaining the largest proportions of observed resting-state MEG beta waveforms in post central gyrus. FIGS. 6(A) and 6(B) respectively illustrate sparse code and data reconstruction for a single trial. Sparse code rows correspond to atoms in FIGS. 5(A)-5(D). FIG. 7 illustrates a raster of learned atoms within beta event span across trials. Atoms aligned along the beta event trough may be used to estimate the trough position. Next, as shown in FIG. 8, peak and trough positions in observed beta events have been identified by the computing server 114 of the present disclosure using the atoms' extreme and locations in the sparse code. FIG. 9 shows a raster of detected peaks and troughs within beta event span across multiple trials. Redundant extrema produced by overlapping atoms may be removed.

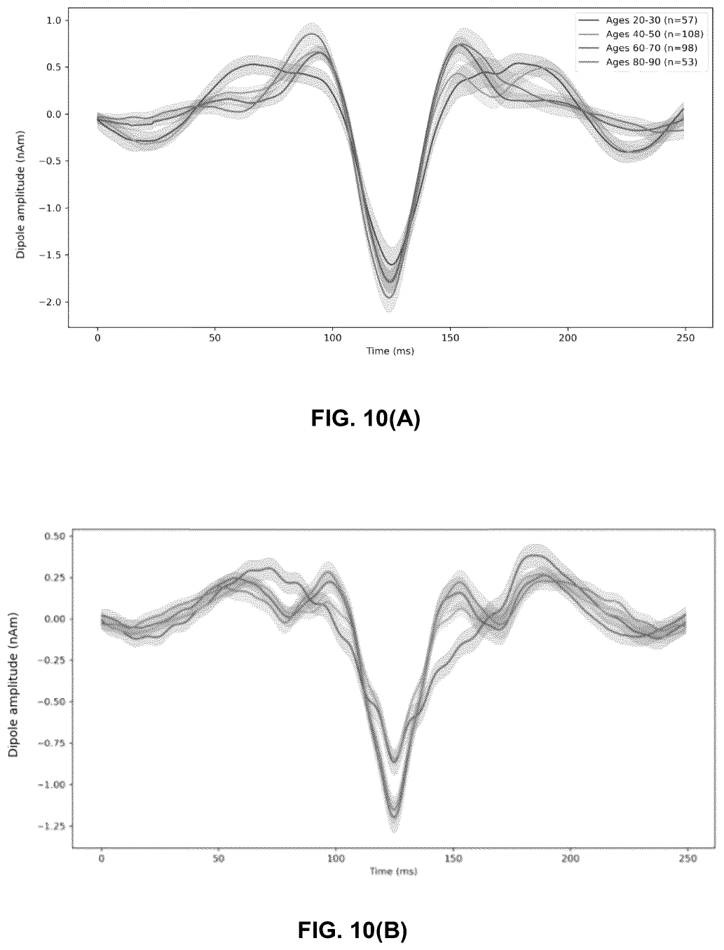

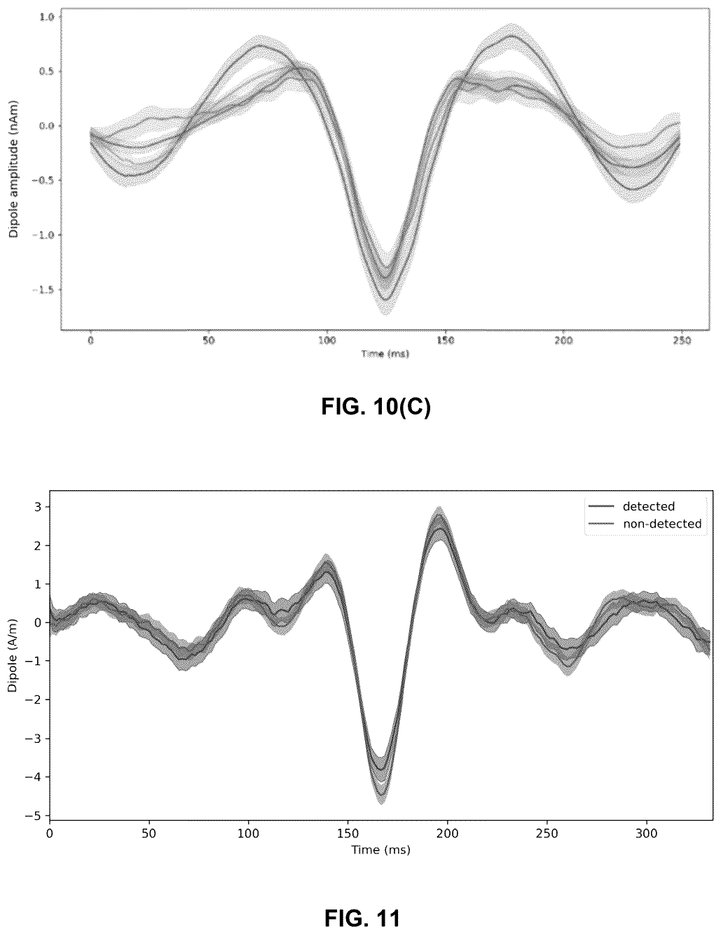

FIGS. 10(A), 10(B), 10(C), 11, and 12 respectively illustrate a number of mean waveforms aligned by troughs detected by the computing server 114 of the present disclosure at different subject or group levels.

Specifically, for four different age deciles (ages 20-30 (n=57); ages 40-50 (n=108); ages 60-70 (n=98); ages 80-90 (n=53)), FIGS. 10(A)-10(C) respectively show mean resting-state beta waveforms in four brain regions aligned by central troughs detected from subject-level, multi-cycle. FIG. 10(A) corresponds to postcentral gyrus, FIG. 10(B) corresponds to caudal anterior cingulate, and FIG. 10(C) corresponds to precuneus. Individual subject waveforms may be aligned by temporal positions assigned to at least one of extracted feature types of the beta wave events to indicate a brain state. The analysis of the present disclosure reveals age-related shifts in the beta event waveform shape, and beta event peaks and troughs increase with health aging.

Beta event peaks and troughs may also increase in non-detected sensory trials. FIG. 11 illustrates mean waveforms aligned by troughs detected from group-level, sub-cycle.

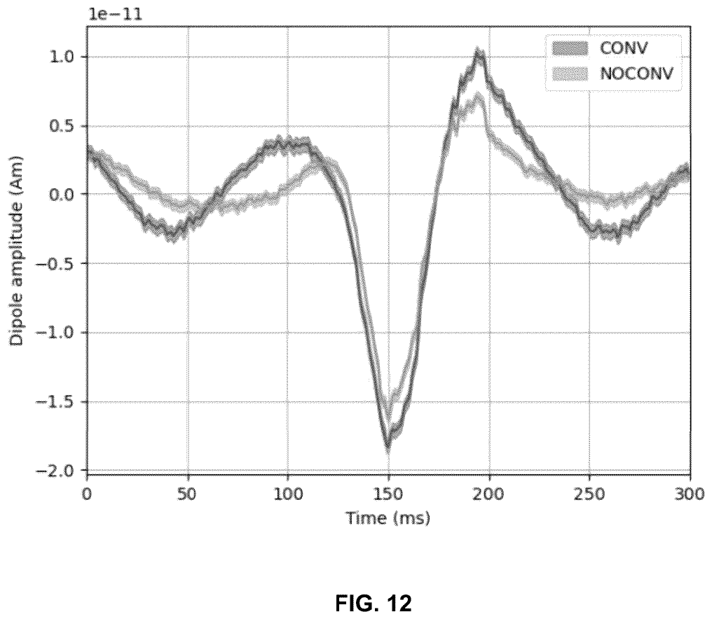

Additionally, beta event peaks and troughs increase in patents who convert to AD. Referring to FIG. 12, beta mean waveforms are aligned by central troughs detected from subject level, multi-cycle (C (convert) group: n=40, NC (not convert) group: n=40).

According to another embodiment, instead of using a global threshold calculated from a dataset on beta amplitude or power, the waveform detection and learning model 246 of the computing server 114 may detect every peak in a beta event above an aperiodic spectrum as a candidate burst event using an iterative algorithm to detect all bursts with amplitudes above the noise floor. As such, bursts across a wider range of amplitudes may be detected, allowing subsequent analyses to determine which ones are functionally relevant. Next, the computing server 114 may apply the principal component analysis to burst waveforms to define a set of dimensions, or motifs, that best explain waveform variance.

In another embodiment, the waveform detection and learning model 246 may be configured to quantify oscillatory features of beta waveforms in the time domain on a cycle-by-cycle basis. Specifically, the waveform detection and learning model 246 may analyze received M/EEG recordings to remove aperiodic and non-oscillatory data by segmenting the recordings into cycles. Putative peaks and troughs may be identified via a series of filtering. Each confirmed oscillatory cycle may be identified by its amplitude, period, and waveform symmetry.

In sum, the system 100 of the present disclosure leverages various MEG datasets and simulation-based neural modeling to examine the age-related alternations in waveform shapes, and to uncover cellular and circuit neurophysiology underlying these changes. Using a data-driven multiscale neural modeling approach, the system 100 may be configured to quantify waveform signatures of aging in resting beta and evoked MEG recordings and determine cell and circuit parameters explaining aging-related waveform changes. Age-related changes in stereotypical waveform features of both beta oscillations and evoked waveforms persist across subjects and reflect systematic alterations in inhibition, synchrony, and connectivity of pyramidal neurons within canonical circuits of the neocortex, thereby explaining age-related changes in MEG activity. In some aspects, the present disclosure may identify and validate reliable age-related beta waveform biomarkers in resting state by analyzing beta event waveforms to determine whether they exhibit stereotyped, quantifiable characteristics across subjects, including trough depth and sharpness, using novel waveform analysis methods. Moreover, the present disclosure may characterize auditory processing dynamics during mismatch response tasks in healthy aging subjects. For example, the system 100 may be configured to characterize aging-related waveform changes in the MMN signature using novel waveform analysis methods to determine whether there are quantifiable aging-related changes in the timing and amplitude of peaks in the MMN evoked response.

In accordance with additional aspects, the system 100 of the present disclosure may use ERP to capture neural activity related to sensory and cognitive processes. ERPs are very small voltages generated in a subject's brain in response to specific events or stimuli. ERPs may reflect the summed activity of postsynaptic potentials produced when a large number of similarly oriented cortical pyramidal neurons (in the order of thousands or millions) fire in synchrony while processing information.

Generally, ERPs may be divided into 2 categories. The early waves, or components peaking roughly within the first 100 milliseconds after stimulus, are termed “sensory” or “exogenous” as they depend largely on the physical parameters of the stimulus. In contrast, ERPs generated in later parts reflect the manner in which the subject evaluates the stimulus and are termed “cognitive” or “endogenous” ERPs, as they examine information processing. The waveforms are described according to latency and amplitude.

Similar to beta waveforms, ERP waveforms may include a series of positive and negative voltage deflections (peaks and troughs), which are related to a set of underlying components. Though some ERP components are referred to with acronyms (e.g., contingent negative variation (CNV), error-related negativity (ERN)), most components are referred to by a letter (N/P) indicating polarity (negative/positive), followed by a number indicating either the latency in milliseconds or the component's ordinal position in the waveform. For instance, a negative-going peak that is the first substantial peak in the waveform and often occurs about 100 milliseconds after a stimulus is presented is often called the N100 (indicating its latency is 100 ms after the stimulus and that it is negative) or N1 (indicating that it is the first peak and is negative); it is often followed by a positive peak, usually called the P200 or P2. The stated latencies for ERP components are often quite variable, particularly so for the later components that are related to the cognitive processing of the stimulus. For example, the P300 component may exhibit a peak anywhere between 250-700 ms.

Referring back to FIGS. 1 and 2, the data acquisition system 104 of the present disclosure may be configured to include an ERP module (not shown) to obtain ERP data from at least one individual 102 and transmit the data to the computing device 212 and/or the backend computer server system 114 for analyzing detected waveform features of the data to predict clinical indications or functional brain states, similar to the aspects discussed above with respect to beta waveforms.

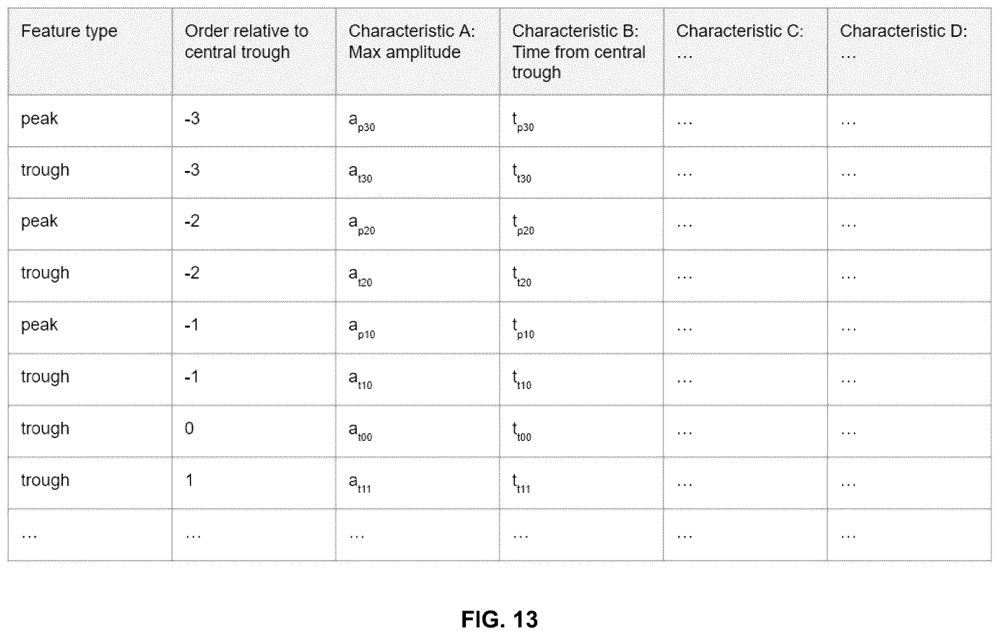

FIG. 13 further illustrates a table of features extracted from a given feature extraction technique and their respective characteristics, including amplitude and timing relative to a central trough, in accordance with aspects of the present disclosure.

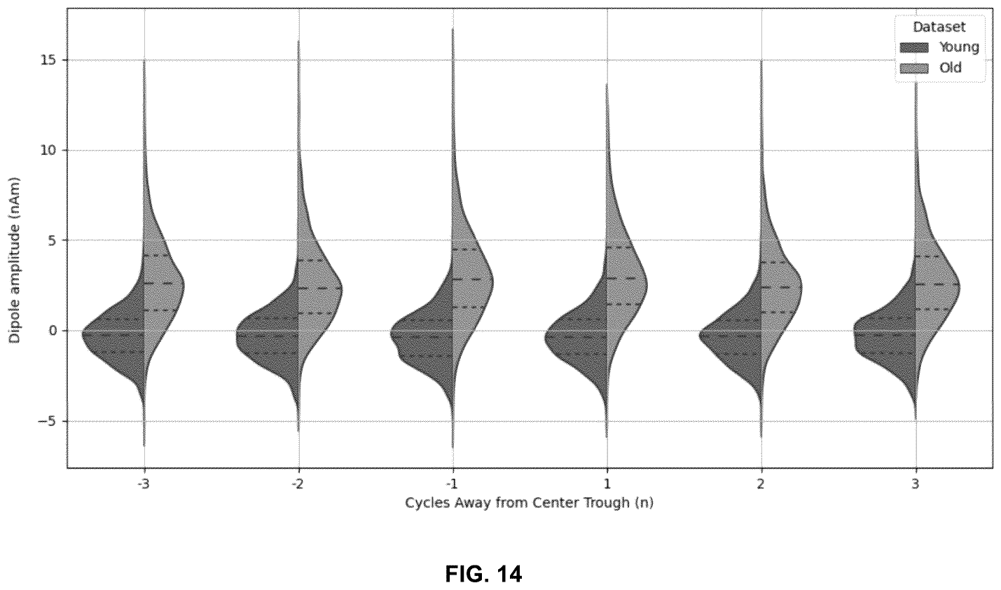

Referring to FIG. 14, empirical distributions of amplitudes of peak features categorized by their order relative to a central trough demonstrate a strong statistical difference between representative young and old subjects, according to an exemplary aspect of the present disclosure.

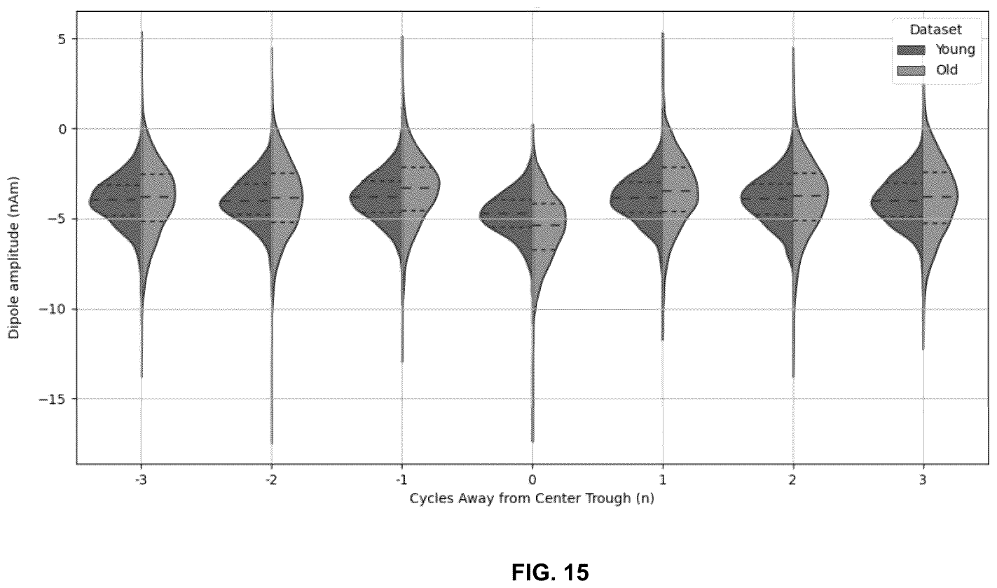

FIG. 15 additionally illustrates empirical distributions of amplitudes of trough features categorized by their order relative to a central trough demonstrating a weak statistical difference between representative young and old subjects.

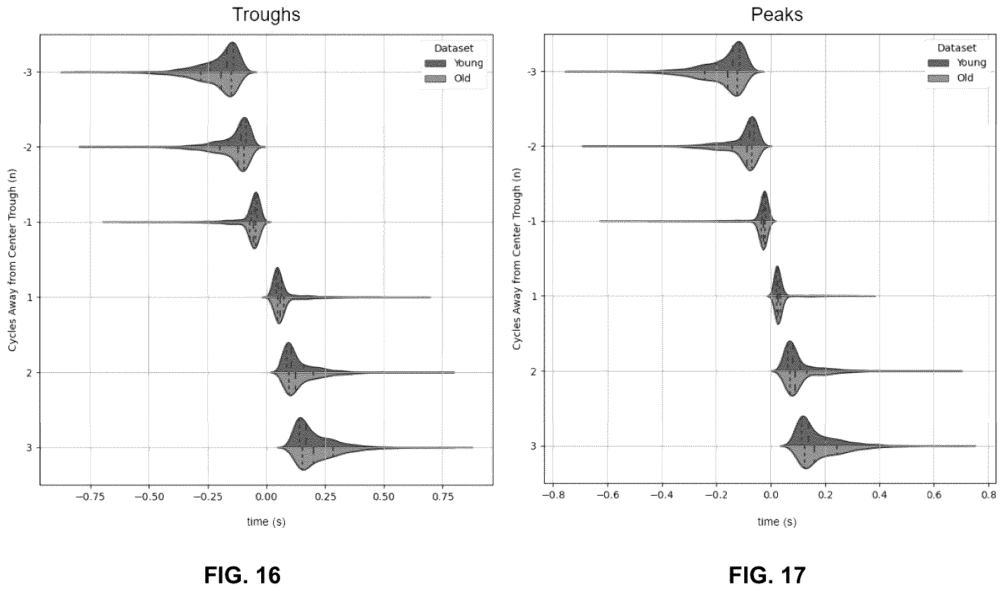

Moreover, referring to FIGS. 16 and 17, empirical distributions of trough and peak timing features relative to a central trough and categorized by their order relative to the central trough demonstrate varying degrees of statistical differences between representative young and old subjects.

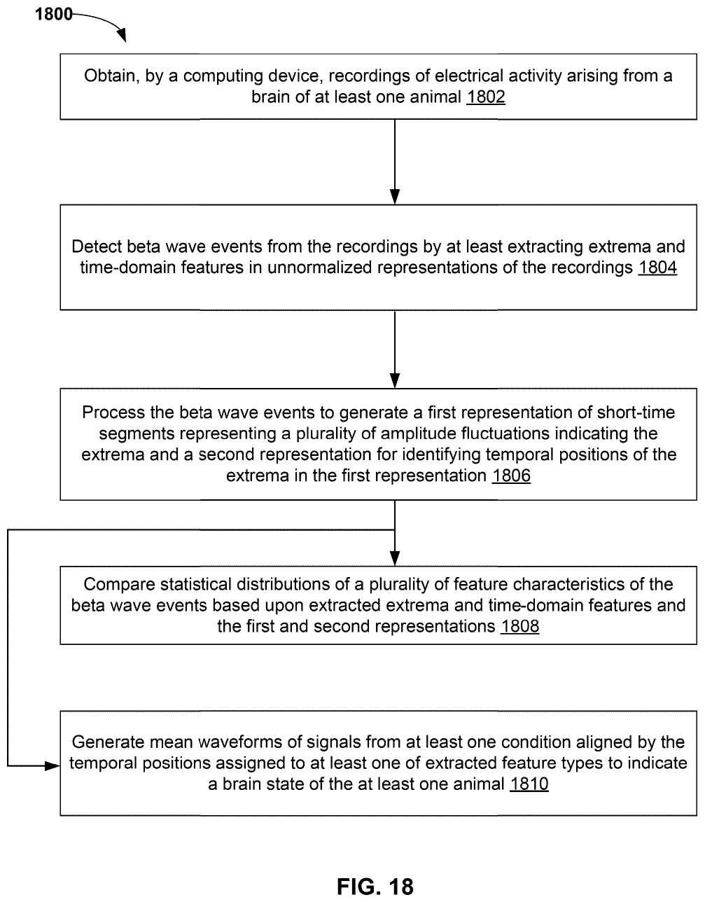

According to aspects of the present disclosure, FIG. 18 illustrates a flowchart of a feature learning and extraction computer-implemented method 1800 for detecting and analyzing non-stationary, transient, or locally structured signals from longer time-series data of obtained beta waveforms of an animal to characterize a biomarker of a brain state or condition. Method 1800 may comprise obtaining (1802) recordings of electrical activity arising from a brain of at least one animal and detecting (1804) beta wave events from the recordings (e.g., Morlet wavelet decomposition or other detection methods) by at least extracting extrema and time-domain features in unnormalized representations of the recordings. For example, the recordings may comprise at least one of M/EEG recordings, LFP recordings, or ECOG recordings. In one aspect, method 1800 may include processing (1806) the beta wave events (e.g., using time-domain feature extraction techniques described above) to generate a first representation of short-time segments representing a plurality of amplitude fluctuations indicating the extrema and a second representation for identifying temporal positions of the extrema in the first representation. In some embodiments, method 1800 may include comparing (1808) statistical distributions of a plurality of feature characteristics (extrema amplitudes, extrema timing, etc.) of the beta wave events based upon extracted extrema and time-domain features and the first and second representations. In other embodiments, method 1800 may include generating (1810) mean waveforms of signals from at least one condition aligned by the temporal positions assigned to at least one of extracted feature types (e.g., central troughs of mean beta waveforms) to indicate a brain state of the at least one animal.

It should be appreciated that the present disclosure may be used to detect alpha events (approximately 7-14 Hz) and gamma events (approximately 30-54 Hz) based on obtained recordings of electrical activity arising from a brain of an animal, similar to the aspects discussed above with respect to beta wave events detection and analysis.

Unless specifically stated otherwise as apparent from the foregoing disclosure, it is appreciated that, throughout the present disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

As used herein, the term “comprising” is not intended to be limiting, but may be a transitional term synonymous with “including,” “containing,” or “characterized by.” The term “comprising” may thereby be inclusive or open-ended and does not exclude additional, un-recited elements or method steps when used in a claim. For instance, in describing a method, “comprising” indicates that the claim is open-ended and allows for additional steps. In describing a device, “comprising” may mean that a named element(s) may be essential for an embodiment or aspect, but other elements may be added and still form a construct within the scope of a claim. In contrast, the transitional phrase “consisting of” excludes any element, step, or ingredient not specified in a claim. This is consistent with the use of the term throughout the specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material. None is admitted to be prior art.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1. A system deployed within a communication network, the system comprising:
a first computing device comprising:
a non-transitory machine-readable storage medium storing instructions; and
a processor coupled to the non-transitory computer-readable storage medium and configured to execute the instructions to:
obtain recordings of electrical activity arising from a brain of at least one animal,
detect beta wave events from the recordings by at least extracting extrema and time-domain features in unnormalized representations of the recordings,
process the beta wave events to generate a first representation of short-time segments representing a plurality of amplitude fluctuations indicating the extrema and a second representation for identifying temporal positions of the extrema in the first representation,
compare statistical distributions of a plurality of feature characteristics of the beta wave events based upon extracted extrema and time-domain features and the first and second representations, and
generate mean waveforms of signals from at least one condition aligned by the temporal positions assigned to at least one of extracted feature types to indicate a brain state of the at least one animal.

2. The system of claim 1, further comprising a second computing device configured to collect the recordings of the at least one animal via a plurality of sensors, wherein the recordings comprise at least one of magneto- or electroencephalograph (M/EEG) recordings, local field potential (LFP) recordings, or electrocorticogram (ECoG) recordings.

3. The system of claim 1, wherein the extrema and time-domain features comprise characteristics selected from the group consisting of: a peak time, a trough time, a peak amplitude, a trough amplitude, an oscillation period, a peak width, a trough width, an inter-peak timing, and an inter-trough timing.

4. The system of claim 3, wherein the processor is further configured to execute the instructions to:
determine empirical distributions of the characteristics; and
simulate, via resampling, an effect of alterations of empirical distributions of at least one of the characteristics on the mean waveforms.

5. The system of claim 1, wherein the processor is further configured to execute the instructions to:
store normative data of waveform features of the beta wave events and empirical or probability distributions or likelihoods thereof, and
employ one or more time-domain feature learning and extraction methods to perform prediction of clinical indication and functional brain states of the at least one animal, wherein the one or more time-domain feature learning and extraction methods based upon at least one of: convolutional dictionary learning (CDL), convolutional sparse coding, translation invariant dictionary learning, cycle-by-cycle analysis, phase estimation or Hilbert transform methods, sliding window matching, template matching, empirical mode decomposition, recurrent neural networks or time delay neural networks or convolutional neural networks trained on features or short segments, dynamic time warping, motif learning or discovery, adaptive time-domain signal processing, temporal representation learning, or one or more unsupervised or semi-supervised machine learning algorithms.

6. The system of claim 1, wherein the extrema and time-domain features include a plurality of peaks and troughs between rising and falling zero-crossings of the beta wave events, wherein the at least one of the extrema includes a plurality of central troughs detected from the beta wave events.

7. The system of claim 1, wherein the processor is further configured to execute the instructions to display the mean waveforms aligned by at least one of the extrema to inform predictive biomarkers and targeted interventions.

8. A computer-implemented method comprising:
obtaining, by a processor of a first computing device, recordings of electrical activity arising from a brain of at least one animal;
detecting, via the processor, beta wave events from the recordings by at least extracting extrema and time-domain features in unnormalized representations of the recordings;
processing, via the processor, the beta wave events to generate a first representation of short-time segments representing a plurality of amplitude fluctuations indicating the extrema and a second representation for identifying temporal positions of the extrema in the first representation;
comparing, via the processor, statistical distributions of a plurality of feature characteristics of the beta wave events based upon extracted extrema and time-domain features and the first and second representations; and
generating, via the processor, mean waveforms of signals from at least one condition aligned by the temporal positions assigned to at least one of extracted feature types to indicate a brain state of the at least one animal.

9. The computer-implemented method of claim 8, further comprising collecting, by a second computing device, the recordings of the at least one animal via a plurality of sensors, wherein the recordings comprise at least one of magneto- or electroencephalograph (M/EEG) recordings, local field potential (LFP) recordings, or electrocorticogram (ECoG) recordings.

10. The computer-implemented method of claim 8, wherein the extrema and time-domain features comprise characteristics selected from the group consisting of: a peak time, a trough time, a peak amplitude, a trough amplitude, an oscillation period, a peak width, a trough width, an inter-peak timing, and an inter-trough timing.

11. The computer-implemented method of claim 10, further comprising:
determining, via the processor, empirical distributions of the characteristics; and
simulating, via resampling, an effect of alterations of empirical distributions of at least one of the characteristics on the mean waveforms.

12. The computer-implemented method of claim 8, further comprising:
storing, via the processor, normative data of waveform features of the beta wave events and empirical or probability distributions or likelihoods thereof; and
employing, via the processor, one or more time-domain feature learning and extraction methods to perform prediction of clinical indication and functional brain states of the at least one animal, wherein the one or more time-domain feature learning and extraction methods based upon at least one of: convolutional dictionary learning (CDL), convolutional sparse coding, translation invariant dictionary learning, cycle-by-cycle analysis, phase estimation or Hilbert transform methods, sliding window matching, template matching, empirical mode decomposition, recurrent neural networks or time delay neural networks or convolutional neural networks trained on features or short segments, dynamic time warping, motif learning or discovery, adaptive time-domain signal processing, temporal representation learning, or one or more unsupervised or semi-supervised machine learning algorithms.

13. The computer-implemented method of claim 8, wherein the extrema and time-domain features include a plurality of peaks and troughs between rising and falling zero-crossings of the beta wave events, wherein the at least one of the extrema includes a plurality of central troughs detected from the beta wave events.

14. The computer-implemented method of claim 8, further comprising displaying, via the processor, the mean waveforms aligned by at least one of the extrema to inform predictive biomarkers and targeted interventions.

15. A non-transitory machine-readable medium storing machine executable instructions for a computing server system, the machine executable instructions being configured for:
obtaining, by a processor of a first computing device, recordings of electrical activity arising from a brain of at least one animal;
detecting, via the processor, beta wave events from the recordings by at least extracting extrema and time-domain features in unnormalized representations of the recordings;
processing, via the processor, the beta wave events to generate a first representation of short-time segments representing a plurality of amplitude fluctuations indicating the extrema and a second representation for identifying temporal positions of the extrema in the first representation;
comparing, via the processor, statistical distributions of a plurality of feature characteristics of the beta wave events based upon extracted extrema and time-domain features and the first and second representations; and
generating, via the processor, mean waveforms of signals from at least one condition aligned by the temporal positions assigned to at least one of extracted feature types to indicate a brain state of the at least one animal.

16. The non-transitory machine-readable medium of claim 15, further comprising instructions for collecting, by a second computing device, the recordings of the at least one animal via a plurality of sensors, wherein the recordings comprise at least one of magneto- or electroencephalograph (M/EEG) recordings, local field potential (LFP) recordings, or electrocorticogram (ECoG) recordings.

17. The non-transitory machine-readable medium of claim 15, wherein the extrema and time-domain features comprise characteristics selected from the group consisting of: a peak time, a trough time, a peak amplitude, a trough amplitude, an oscillation period, a peak width, a trough width, an inter-peak timing, and an inter-trough timing.

18. The non-transitory machine-readable medium of claim 17, further comprising instructions for:
determining, via the processor, empirical distributions of the characteristics;
simulating, via resampling, an effect of alterations of the empirical distributions of at least one of the characteristics on the mean waveforms; and
displaying, via the processor, the mean waveforms aligned by at least one of the extrema to inform predictive biomarkers and targeted interventions.

19. The non-transitory machine-readable medium of claim 15, further comprising instructions for:
storing normative data of waveform features of the beta wave events and empirical or probability distributions or likelihoods thereof; and
employing, via the processor, one or more time-domain feature learning and extraction methods to perform prediction of clinical indication and functional brain states of the at least one animal, wherein the one or more time-domain feature learning and extraction methods based upon at least one of: convolutional dictionary learning (CDL), convolutional sparse coding, translation invariant dictionary learning, cycle-by-cycle analysis, phase estimation or Hilbert transform methods, sliding window matching, template matching, empirical mode decomposition, recurrent neural networks or time delay neural networks or convolutional neural networks trained on features or short segments, dynamic time warping, motif learning or discovery, adaptive time-domain signal processing, temporal representation learning, or one or more unsupervised or semi-supervised machine learning algorithms.

20. The non-transitory machine-readable medium of claim 15, wherein the extrema and time-domain features include a plurality of peaks and troughs between rising and falling zero-crossings of the beta wave events, wherein the at least one of the extrema includes a plurality of central troughs detected from the beta wave events.
